(19) United States
(12) Patent Application Publication
(10) Pub. No.: US 2010/0248962 A1
(43) Pub. Date: Sep. 30, 2010

(54) COMPOSITIONS FOR THE MASKING OF MALODOR IN AGRICULTURAL COMPOSITIONS AND METHODS OF MAKING AND USING THEREOF

(75) Inventor: Kimberly A. WILCZYNSKI, Dublin, OH (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SMG Brands, Inc., Wilmington, DE (US)

(21) Appl. No.: 12/751,492

(22) Filed: Mar. 31, 2010

Related U.S. Application Data
(60) Provisional application No. 61/165,279, filed on Mar. 31, 2009.

(51) Int. Cl. A01N 31/00; A61L 9/01

(52) U.S. Cl. 504/101; 504/354

(57) ABSTRACT

Compositions for masking the odor of malodorous agricultural products comprise at least one odor-masking agent in an amount effective to reduce the malodor. Methods of controlling malodorous components in agricultural products comprise adding an odor-masking agent to the agricultural products. Advantageous odor-masking agents include “green notes” and are surprisingly and particularly adapted for the masking of phenoxy or phenolic odors such as those produced by the herbicide 2,4-D.

This application claims priority to U.S. Provisional application 61/165,279, filed Mar. 31, 2009, the subject matter of which is incorporated herein in its entirety.

### BACKGROUND OF THE INVENTION

1. Field of the Invention

The present invention relates to compositions for reducing malodor impression. More particularly, the present invention relates to compositions designed to reduce malodor in dry agricultural compositions. These compositions may also be used in methods of controlling malodor impression.

2. Background and Description of Related Art

Chemical compositions, especially when concentrated, have a tendency to emit odor. Some odors may be pleasant, while others are distinctly unpleasant. Odors are generally described as sweet, fruity, putrefaction-like, spicy, burned, paint-like, or some combination thereof. Odors may also be grouped into olfactive families. Traditional categories include “single florals” that include the scent produced by a single, particular flower; “floral bouquets” containing the combination of several flowers in a scent; “amber” scents which include the sweet, slightly animalic scents of ambergis or labdanum; “woody” scents such as agarwood, sandalwood, cedar, patchouli (which has camphoraceous tones); “leather” scents which include honey, tobacco, wood, and wood tars; “chypre” scents which include bergamot, oakmoss, patchouli, and labdanum; “fougére” scents which include lavender, coumarin, oakmoss; bright florals; “green” scents which are typically described as a lighter and more “modern” interpretation of the chypre type having cut grass and cucumber like scents; aquatic scents; citrus scents; fruity scents; and gourmand scents which have edible or dessert like odors. Odors may also be described by reference to a fragrance wheel which broadly categorizes scents into groups and subgroups:

| | | |
|---|---|---|
| | Floral | Floral |
| | | Soft Floral |
| | | Floral Oriental |
| | Oriental | Soft Oriental |
| [symbol] | | Oriental |
| | | Woody Oriental |
| Fougère | Woody | Woods |
| | | Mossy Woods |
| [symbol] | | Dry Woods |
| | Fresh | Citrus |
| | | Fruity |
| | | Green |
| | | Water |

Scents categorized in a particular family may comprise undertones or subtle notes of other families. Therefore, a scent which is described as green, for example, may comprise undertones of wood, moss, or any other family.

Many individuals associate particular smells with the “health” or “safety” of a product. As a result, compositions which comprise pungent or distinctly chemical odors are often considered dangerous. Consumers are less likely to purchase a product if they believe its use is hazardous. Chemical odor and safety, however, are not necessarily correlated. Phosgene, a deadly chemical weapon used during World War I, is reported as having the smell of freshly cut grass. In contrast, the popular herbal supplement Valerian (Valeriana officinalis) has a pungent aroma, but is reported as safe for use in treating insomnia. There is a need, therefore, for compositions which inhibit, mask, conceal, or otherwise modulate undesirable odors belonging to consumer-safe products.

A wide variety of deodorizing compositions are known in the art which function either by modifying odors or masking them. Odor modification, in which the odor is changed, e.g., by chemical modification, has been used in many applications. Current malodor modification methods known in the art include oxidative degradation, which uses oxidizing agents such as oxygen bleaches, chlorine, chlorinated materials such as sodium hypochlorite, chlorine dioxide, etc., and potassium permanganate to reduce malodor, and reductive degradation which uses reducing agents such as sodium bisulfate to reduce malodor. Both of these methods are unacceptable for use in agricultural compositions such as fertilizers or herbicides that must retain their original chemical integrity to remain effective.

In another class of compositions, odorants provide an odor which conceals, masks, or inhibits the recognition of malodors. Appropriate perfume ingredients or fragrances need to be selected to maximally inhibit or conceal the odor of particular chemicals. Compositions may also need to be adapted to not only conceal malodors but to connote certain odor qualities such as freshness.

The masking effect of odor-masking agents may be provided by a number of different mechanisms. In a first mechanism, the masking fragrance blends with the malodor compound or compounds to provide a different and more desirable aroma. In a second mechanism, the masking fragrance is employed in a large quantity to overwhelm the olfactory sense. Combinations of these mechanisms may be also be applied.

Manure odor control has been described. Most of the odor from animal manure/urea is generated from the conversion of ammonium (odorless) to ammonia gas and airborne nitrates and sulfates upon the degradation of the manure. An estimated half to three-fourths of the nitrogen in manure from beef cattle feedlots breaks down to ammonia gas and other compounds before it ever reaches farm fields. See USDA Press Release, Glickman Says Research May Help Curb Manure Odor, By Ben Hardin, Dec. 14, 1998. Most methods of odor control in agricultural compositions are directed to the masking or inhibition of odor resulting from the degradation of natural fertilizer products such as manure.

In one method, zeolites have been used to absorb moisture and ammonium odor by capturing water in empty pore spaces and by trapping of the ammonium cation in the mineral lattice.

Other methods attempt to inhibit bacterial growth. For example, U.S. Pat. No. 6,159,262 describes manure fertilizers comprising odor-control and antibacterial agents. These agents include o-dichlorobenzene, enzymes, “natural products” including extracts of pine or lemon, and even “Australian essential oils” including eucalyptus oil, tea tree oil, banksia oil, and others. The listing of plant species from which natural oils may be obtained is hereby incorporated by reference. U.S. Pat. App. No. 2002/0134315 describes the use of western juniper to mask the odor of pesticide materials.

Reactive odor control agents have also been described. U.S. Pat. No. 4,997,469 describes the addition of aldehydes, such as formaldehyde, methylolurea, urea-formaldehyde concentrate, methylene urea, hexamethylene tetramine, acetaldehyde, crotonaldehyde, and propionaldehyde, to eliminate the odor of certain nitrogenous compounds including manure based compounds, presumably by reaction of the aldehyde with the nitrogen compound.

Despite these disclosures, satisfactory odorants for compounding with agricultural products, particularly non-manure fertilizer products whose odor generally cannot be controlled by modulation of bacterial growth, chemical decomposition, or zeolite trapping, are still needed. Furthermore, desirable and advantageous odorant compositions must be substantially chemically inactive with respect to the active ingredients of the agricultural product, must be safe for consumer use, and must mask or conceal pungent or chemical odors. Other advantageous properties of desirable odorant compositions may include the production of pleasant odors, long-term odor masking maintenance, the ability to be directly compounded into an agricultural composition, or the ability to be sprayed onto packaging or agricultural compositions.

### SUMMARY OF THE INVENTION

The present invention, in one or more embodiments, is a composition for reducing malodor impression. The composition may comprise a perfume or fragrance and may be further adapted to conceal particular odors present in agricultural compositions such as fertilizers, herbicides, insecticides, fungicides, or any combination thereof.

In one embodiment, the invention is a composition, comprising a dry agricultural composition, comprising a phenolic or phenoxy-like malodor; and an amount of at least one odor-masking agent effective to reduce said malodor, comprising phenyl ethyl methyl ether and optionally galbanum oil, phenyl acetaldehyde, phenyl acetaldehyde dimethyl acetal, cis-3-hexenyl acetate, cinnamic alcohol, or any combination thereof. The malodor may be produced at least in part by a phenoxy compound, a halophenoxy compound, or a chlorophenoxy compound, such as (2,4-dichlorophenoxy)acetic acid. The agricultural composition may comprise at least one herbicide, a chlorophenoxy herbicide, a pesticide, a fertilizer, or a combination thereof. In a preferred sub-embodiment, the composition produces an odor characterized as pleasant-smelling or neutral. The amount of odor-masking agent may be adjusted depending on the strength of the malodor and can, for example, comprise between about 0.01% and about 1% by weight of the composition, alternatively between about 0.025% to about 0.5%, further alternatively between about 0.05% to about 0.1%, and even further alternatively about 0.05% or about 0.07%. Various agricultural compositions and formulations may be used. In one embodiment, the agricultural composition is granulated and has an average seive size of about −10/+40. In preferred embodiments the odor-masking agent comprises a combination of galbanum oil, phenyl ethyl methyl ether, phenyl acetaldehyde, phenyl acetaldehyde dimethyl acetal, cis-3-hexenyl acetate, and cinnamic alcohol. The composition may comprise an inner core and an outer core, wherein said inner core comprises said agricultural composition and said outer core comprises said odor-masking agent.

In another embodiment, the invention is a composition, comprising a dry agricultural composition, comprising at least one phenolic or phenoxy-like odor; and an amount of at least one odor-masking agent effective to reduce at least one phenolic or phenoxy-like odor. The combination of said phenolic or phenoxy-like odor and the odor of said odor-masking agent may comprise one or more green notes and further, the combination may result in the reduction of phenolic or phenoxy-like odor. Preferably, the composition comprises green notes. The green notes may be characterized as earthy, like green apple, fresh, like balsamic, woody, spicy, warm, floral, sweet, like hyacinth, like clover, like honey, like cocoa, like foliage, rosy, like banana, or any combination thereof. The green notes may also be substantially the result of the odor of phenyl ethyl methyl ether. The odor-masking agent may combine with the malodor to produce green notes characterized as fresh, hyacinth-like, rosy, or green-apple like.

In another embodiment, the invention is a method for reducing malodor in an agricultural composition, comprising the step of adding at least one odor-masking agent to a dry agricultural composition comprising a malodor, wherein said odor-masking agent comprises phenyl ethyl methyl ether and optionally galbanum oil, phenyl acetaldehyde, phenyl acetaldehyde dimethyl acetal, cis-3-hexenyl acetate, cinnamic alcohol, or any combination thereof. The malodor may be characterized as phenoxy-like or phenolic or as smelling like smells like (2,4-dichlorophenoxy)acetic acid.

In yet another embodiment, the invention is a method of making an agricultural composition with a reduced malodor impression, comprising the step of combining a granulated, dry agricultural composition with an amount of at least one odor-masking agent effective to reduce at least one phenolic or phenoxy-like odor, wherein said odor-masking agent comprises phenyl ethyl methyl ether and optionally galbanum oil, phenyl acetaldehyde, phenyl acetaldehyde dimethyl acetal, cis-3-hexenyl acetate, cinnamic alcohol, or any combination thereof. The method may comprise coating or spraying the odor-masking agent onto the agricultural composition. The malodor may again be characterized as phenoxy-like or phenolic or as smelling like (2,4-dichlorophenoxy)acetic acid.

The present invention is also, in one or more embodiments, a composition, comprising (i) an agricultural composition; and (ii) at least one odor-masking agent. Furthermore, one or more embodiments of the present invention include a method, comprising the step of coating or mixing an agricultural composition with at least one odor-masking agent. In yet other embodiments, the invention is a method for modifying or enhancing the odor of an agricultural composition product which comprises adding an olfactory effective amount of at least one odor-masking agent.

In view of the long felt need to provide compositions that mask, conceal, or inhibit odor that are suitable for use in agricultural products, it is one object of the present invention to provide odorant compositions.

It is a further object of the present invention, in one or more embodiments, to provide odorants which are chemically inert with the agricultural composition. Compositions according to one or more embodiments of the present invention comprise odorants which are consumer friendly: odorants which are safe and which optionally impart a pleasant or neutral odor relative to any malodor resulting from the components of the composition.

It has now been discovered that compositions comprising certain fragrances are uniquely adapted to mask the odors of agricultural products.

More specifically, odorant compositions in accordance with one or more embodiments of the present invention comprise ingredients known as “green notes,” more specifically (but not limited to) galbanum oil, phenyl ethyl methyl ether, phenyl acetaldehyde, phenyl acetaldehyde dimethyl acetal, cis-3-hexenyl acetate, and cinnamic alcohol. Additional natural or essential oils may be added to provide additional “notes” to the composition, although it is preferred that the predominant note is a green note as these notes are advantageous in reducing the malodor impression of phenoxy or phenolic odors.

Exemplary of one or more of the advantages resulting from use of the fertilizer compositions of the present invention are the following:

(1) Odorants can, in one or more embodiments, be added to an agricultural product during the granulation process.

(2) Odorants are, in one or more embodiments, chemically inert to the agricultural ingredients and may also be generally non-hazardous to the consumer.

(3) The odorant effectively conceals malodors resulting from components in an agricultural composition without inhibiting the efficacy of the agricultural composition.

Certain terms of art are used in the specification that are to be accorded their generally accepted meaning within the relevant art; in particular, the following well-understood terms are hereby more fully described.

“Agricultural composition” is a composition useful in the growing of plants or the maintenance of a natural environment, e.g., a garden or lawn, and may comprise, without limitation, fertilizers, acaricides, avicides, bactericides, biocides, germicides, rodenticides, vulpicides, insecticides, fungicides, herbicides, microbicides, defoliants, pH adjustors, soil conditioners, molluscicides, pesticides, or any combination thereof. Such compositions may be used in commercial agricultural settings, households and home-gardens, general horticulture, and other venues.

“Dry” means the component, composition or mixture comprises less than 10% w/w water.

“Effective amount of an odor-masking agent” is an amount of odor-masking agent which results in an appreciable reduction of malodor impression.

“Fertilizer composition” denotes a composition comprising nutrients for plants.

“Fragrance precursor” denotes a substance or mixture of chemicals which upon addition to a substrate, substance or mixture of chemicals provides an odor (or different odor) detectable by the olfactory sense of a human. A fragrance precursor may or may not be generally and qualitatively described as pleasant or neutral. A fragrance precursor may or may not have an odor.

“Fragrance” denotes a substance or mixture of chemicals detectable by the olfactory sense of a human. Unless otherwise defined, a fragrance denotes odors generally and qualitatively described as pleasant or neutral.

“Green notes” or “green odors” are smells described as green in the art of perfumery or fragrance formulation. As used herein, green notes or green odors comprise a predominantly green smell but may also be described as comprising the following notes: earthy, green apple or Granny Smith apple, fresh, balsamic, woody, spicy, warm, floral, sweet, hyacinth, clover, honey, cocoa, foliage, metal, rosy, banana notes, and any combination of notes thereof. More particularly, green notes or green odors include odors which result from the compounds galbanum oil, phenyl ethyl methyl ether, phenyl acetaldehyde, phenyl acetaldehyde dimethyl acetal, cis-3-hexenyl acetate, cinnamic alcohol, or any combination thereof, and preferably the combination of all. These green notes—when combined with a target having a phenolic or phenoxy-like odor—surprisingly and advantageously provide an odor impression from the target that is markedly less phenolic or phenoxy-like.

“Malodor impression” denotes the detection by the olfactory sense of a human of an odor which is qualitatively and generally perceived as offensive or unpleasant.

“Malodor reduction” denotes the reduction in the detectability of a malodor, whether by desensitization or overloading of the olfactory sense, chemical degradation or transformation of the odor-causing component or composition, or modification of the qualitative perception of the odor such as, for example, by combination with a substance to provide a new, distinct and pleasant or non-offensive odor.

“Malodor” denotes an odor that is unpleasant. Other terms which may used to describe an unpleasant odor include “stench,” “reek,” “disgusting,” “stink,” and “raunch.”

“Natural fertilizer composition” denotes fertilizers which are produced by the degradation of animal waste or garbage and which comprises some amount of animal waste or garbage. Animal waste may be excretory waste such as manure.

“Notes” are descriptors of scents that can be sensed upon the application or dispersion of a perfume onto a target. Notes are separated into three classes: top notes, middle/heart notes, and base notes. The classes denote groups of smells that can be sensed with respect to the time after the application of a perfume. The scents that are perceived immediately on application of a perfume are called top notes and usually consist of relatively small, light molecules that evaporate quickly. They form a person's initial impression of a perfume. The scents of this note class are often described as “fresh,” “assertive” or “sharp.” The compounds that contribute to top notes are usually strong in scent, very volatile, and evaporate quickly. Citrus and ginger scents are common top notes. Top notes are also called head notes. Middle notes are the scent of a perfume that emerges just prior to when the top notes dissipate. The middle note compounds form the “heart” or main body of a perfume and emerge in the middle of the perfume's dispersion process. They serve to mask the often unpleasant initial impression of base notes, which become more pleasant with time. The scent of middle note compounds is usually more mellow and “rounded.” Scents from this note class appear anywhere from two minutes to one hour after the application of a perfume. Lavender and rose scents are typical middle notes. They are also called the “heart notes.” Base notes comprise the scent of a perfume that appears close to the departure of the middle notes. The base and middle notes together are the main theme of a perfume. Base notes bring depth and solidity to a perfume. Compounds of this class are often the fixatives used to hold and boost the strength of the lighter top and middle notes. Consisting of relatively large, heavy molecules that evaporate slowly, compounds of this class of scents are typically rich and “deep” and are usually not perceived until about 30 minutes after the application of the perfume to a target or during the period of perfume dry-down. Some base notes can still be detectable in excess of about twenty-four hours after application, particularly the animalic notes. Fragrant materials are listed in order of volatility and are grouped under respective evaporation coefficients (perfume notes) that range from 1 to 100. Top notes, being most volatile, have evaporation coefficients in the range of 1 to 14, middle notes are in the range of 15 to 60, and base notes are in the range of 61 to 100.

“Odor-masking agent” denotes the substance, mixture, or composition which functions to mask odor.

“Odor masking” denotes the concealment of an odor such that a person's perception of the odor is diminished.

“Odor persistence” denotes the length of time a detectable odor can be perceived by the human olfactory sense after “olfactory resting.”

“Odorant” denotes a substance, mixture, or composition which produces an odor.

“Olfactory effective amount” is an amount which is detectable by most persons under normal usage conditions.

“Olfactory resting” denotes the re-sensitization of the olfactory sense after exposure to an odor such as, for example, by removal of the odor from the environment for such a period of time that the same qualitative level of odor for the same exposure to a fixed amount of odor, as measured by the concentration of volatile compounds in a 3D space, is perceived.

“Perfume” denotes a concentrated fragrance. Unless otherwise defined, a perfume is a fragrance having a concentration of at least 10% w/w.

“Pleasant odor” denotes an odor that is generally perceived as non-offensive. Likewise, an “unpleasant odor” denotes an odor that is generally perceived as offensive. An offensive odor is one in which most persons would not desire exposure to.

“Target” is generally used to denote an agricultural compound having a malodor.

“w/w” means weight per weight.

### BRIEF DESCRIPTION OF THE DRAWINGS

FIG. 1 is a graphical representation of the process of making an agricultural composition.

FIG. 2 is a graphical representation of an example of the blending process.

### DETAILED DESCRIPTION

In one embodiment of the present invention, a composition is formulated comprising a fertilizer, an herbicide, and an odor-masking agent, wherein the odor-masking agent is primarily phenyl ethyl methyl ether, also known as chrysanthemum oxide (CAS No. 3558-60-9). In another embodiment, the composition comprises fertilizer and herbicide in a total concentration of about 99.90% to about 99.95% w/w, about 0.1% to about 0.05% w/w odor-masking agent, and other inert compounds to 100%. The odor-masking agent preferably comprises ingredients known as having “green notes,” more specifically (but not limited to) galbanum oil, phenyl ethyl methyl ether, phenyl acetaldehyde, phenyl acetaldehyde dimethyl acetal, cis-3-hexenyl acetate, and cinnamic alcohol.

Green notes have a distinct odor type well known by those skilled in the art of perfumery. Green notes may be described as comprising a green scent or tonality and may also include the following notes: earthy, green apple, fresh, balsamic, woody, spicy, warm, floral, sweet, hyacinth, clover, honey, cocoa, jasmine, metallic, foliage, rosy, banana, and any combination thereof. Green notes of one or more embodiments of the present invention may comprise a complex array of scents, some barely perceptible and others relatively strong, that are preferably a result of a combination of galbanum oil, phenyl ethyl methyl ether, phenyl acetaldehyde, phenyl acetaldehyde dimethyl acetal, cis-3-hexenyl acetate, and cinnamic alcohol. These green notes—when combined with a target having a phenolic or phenoxy-like odor—provide an odor impression from the target that is markedly less phenolic or phenoxy-like.

Surprisingly, it has been discovered that the green notes of the present invention, particularly phenyl ethyl methyl ether, but also including galbanum oil, phenyl acetaldehyde, phenyl acetaldehyde dimethyl acetal, cis-3-hexenyl acetate, and cinnamic alcohol are particularly useful for the masking of phenolic or phenoxy odors such as those found in many agricultural compositions.

In one or more embodiments of the present invention, the odor-masking agent is added in amount sufficient to completely mask a malodor. In other embodiments, the odor-masking agent may be added in amount to substantially mask a malodor. It may be desirable in some instances to retain a hint of malodor in a composition to remind a consumer that the product contains “active” chemical components. Some consumers may even desire a faint chemical smell. Accordingly, in some embodiments, odor-masking agents of the present invention are added in amounts that reduce but do not eliminate a target odor.

Some notes used in the present composition to produce an odor with green notes are listed in Table 1 along with their respective odors. Boiling points are provided as an aid to determining the relative volatilities, and hence evaporation coefficients, of the various ingredients. It is noted that green notes may develop only upon compounding of one scent with another.

TABLE 1
Organoleptics

| Compound | Odor(s) | Boiling Point* |
|---|---|---|
| phenyl ethyl | Green; floral; chrysanthemum; | 185-187 |
| methyl ether | hyacinth; etheral; jasmine; | |
| | metallic; rose; slight fruit | |
| galbanum oil | Fresh; green; cortex; earthy; | 196.6** |
| | rooty; woody; balsam | |
| phenyl | Green; sweet; floral; hyacinth; | 193-195 |
| acetaldehyde | clover; honey; cocoa | |
| phenyl | Green; foliage; floral; rosy; | 219-221 |
| acetaldehyde | earthy; mushroom | |
| dimethyl acetal | | |
| cis-3-hexenyl | Sweet; leafy; green; fresh; | 65 |
| acetate | fruity; waxy; apple | |
| cinnamic alcohol | Sweet; balsam; hyacinth; spicy; | 250-258 |
| | green; powdery; cinnamon | |

*° C. at 760 mm Hg
**Adapted Stein & Brown method. See Handbook of property estimation methods for chemicals: environmental and health sciences; By Robert S. Boethling, Donald Mackay; CRC Press, 2000.

Other compounds which impart odor or odors may also be used. For example, while galbanum oil is a preferred odor, other natural oils having similar odor (either alone or in combination with other notes) may also be used. In one embodiment, substitute compounds effective (either alone or in combination with other odors) to reduce the malodor impression of phenoxy or phenolic odors in a target are used. In another embodiment, substitute compounds that provide (either alone or in combination with any other odor agents) an overall green odor are used. An odor-masking agent which has green notes or green odor may comprise individual odor agents which alone would not be characterized as having a green odor or note. An odor-masking agent has green notes based on the overall sense impression the combination of odor agents imparts.

In one embodiment of the present invention, a granulated agricultural composition is blended with an odor-masking agent to produce a composition with improved odor properties. In other embodiments, the composition produced has a reduced malodor impression.

Suitable methods of producing compositions with improved odor properties, such as reduced malodor impression, may comprise the addition of an odor-masking agent to an agricultural composition. More particularly, the odor masking reagent may comprise phenyl ethyl methyl ether and is, for example, added in a concentration of about 0.1 to about 1.0 w/w %. Such addition may be by blending in a rotary, ribbon, or other blender known in the art of producing agricultural formulations. The odor masking reagent may be added at any stage of the formulation of the composition.

In one embodiment, the odor-masking agent is added prior to the packaging of the composition. For example, the odor masking reagent may be added to a granulated agricultural composition after final addition of active ingredients such as, for example, surfactants, preservatives, and other compounds, but prior to the addition of the composition to packaging.

The agricultural compositions, in one or more embodiments, are fertilizers, insecticides, herbicides, fungicides, or any combination thereof. In one embodiment, the composition is a mixture of one or more fertilizers and one or more herbicides, insecticides, or fungicides. In yet another embodiment, the composition is a mixture of a fertilizer composition and an herbicide.

In alternative embodiments, the odor-masking agent may be added to a dry blend of components prior to granulation or other processing. In yet another alternative embodiment, the odor-masking agent may be added to a wet or liquid blend of components.

An example of an odor source generally considered offensive is the herbicide 2,4-dichlorophenoxyacetic acid (2,4-D; CAS No. 94-75-7). 2,4-D is one of the most widely used herbicides in the United States. Herbicides are chemicals used to control weed growth. 2,4-D belongs to the group of related synthetic herbicides called chlorophenoxy herbicides. The chemical structure of 2,4-D resembles indoleacetic acid, a naturally occurring hormone produced by plants to regulate their own growth. This resemblance allows 2,4-D to artificially regulate plant growth. Some of the common trade names of 2,4-D containing products sold in the U.S. are CHLOROZONE, SALVO, WEED-NO-MORE and AQUA-KLEEN.

The odor from 2,4-D may be described as phenolic or phenoxy-like. Because this odor source is distinctly chemical, consumers may be hesitant to utilize products containing the herbicide in areas where, for example, children or pets play, despite the relative safety of the compound. In one embodiment of the present invention, the odor-masking agent is a composition comprising ingredients known as having “green notes,” more specifically (but not limited to) galbanum oil, phenyl ethyl methyl ether, phenyl acetaldehyde, phenyl acetaldehyde dimethyl acetal, cis-3-hexenyl acetate, and cinnamic alcohol. Surprisingly, this composition is effective at eliminating 2,4-D odor and does not reduce the efficacy of the herbicide. Therefore, consumers are not subjected to 2,4-D odor upon the opening of a package containing 2,4-D or during storage and use.

It is contemplated that other phenoxy compounds besides 2,4-D may be treated with one or more odor-masking agents. These phenoxy compounds include, without limitation, members of the phenoxy family of herbicides, such as 2,4,5-trichlorophenoxyacetic acid (2,4,5-T); 2-methyl-4-chlorophenoxyacetic acid (MCPA); 2-(2-methyl-4-chlorophenoxy)propionic acids (mecoprop, MCPP); 2-(2,4-dichlorophenoxy)propionic acid (dichloroprop, 2,4-DP); and (2,4-dichlorophenoxy)butyric acid (2,4-DB). These phenoxy compounds may be further delimited into halophenoxy compounds such as, and preferably, chlorophenoxy compounds.

In another embodiment of the present invention, the odor-masking agent is comprised within an “all-in-one” granular particle comprised of fertilizer ingredients (such as ammonium sulfate, monoammonium phosphate, potassium sulfate, urea, urea formaldehyde concentrate, etc.) and active ingredients (for example, 2,4-D and/or mecoprop).

Where the odor-masking agent is a combination of ingredients known as having “green notes,” alone or in combination and more specifically (but not limited to) galbanum oil, phenyl ethyl methyl ether, phenyl acetaldehyde, phenyl acetaldehyde dimethyl acetal, cis-3-hexenyl acetate, and cinnamic alcohol, combination with compounds that have a phenolic or phenoxy-like odor (such as 2,4-D), results in a pleasant smelling composition. In a preferred embodiment, an agricultural composition comprises 2,4-D and phenyl ethyl methyl ether. In another preferred embodiment, an agricultural composition comprises 2,4-D and galbanum oil, phenyl ethyl methyl ether, phenyl acetaldehyde, phenyl acetaldehyde dimethyl acetal, cis-3-hexenyl acetate, and cinnamic alcohol.

Other odor-masking agents may also be used, provided the agent is sufficiently volatile to function as an odor-masking agent with respect to the masked odor. Further, the odor-masking agent must reduce the malodor impression of a target having a phenoxy-like or phenolic odor. Other suitable constituents (with CAS numbers in parens) include amyl salicylate (2050-08-0), benzyl acetate (140-11-4), hedione (24851-98-7), phenyl ethyl alcohol (60-12-8), linalool (78-70-6), amyl cinnamic aldehyde (122-40-7), cyclamen aldehyde (103-95-7), dipropylene glycol (25265-71-8), eugenol (97-53-0), hydroxycitronellal (107-75-5), benzyl benzoate (120-51-4), habanolide (34902-57-3), cis-3-hexenol (928-96-1), peru balsum oil (8007-00-9), prenyl salicylate (68555-58-8), and tripla extra (67801-65-4).

Various alcohols, aldehydes, amines, esters, ethers, ketones, lactones, terpenes, thiols, and other compounds meeting these criteria may also be used.

Examples of odor-masking agents include alcohols such as benzy alcohol (almond), ethyl maltol (cooked fruit), furaneol (strawberry), 1-hexanol (woody), cis-3-hexen-1-ol (grassy), and menthol (peppermint).

Aldehydes include acetaldehyde, benzaldehyde (almond), hexanal (grassy), cinnamaldehyde (cinnamon), citral (lemony), cis-3-hexenal (green tomatoes), furfural (burnt oats), neral (lemongrass), vanillin (vanilla).

Amines include indole (small concentrations—jasmine) and substituted pyrazines.

Esters such as ethyl acetate (fruity), ethyl butanoate, ethyl decanoate, ethyl heanoate, ethyl octanoate, fructone (fruity), hexy acetate (fruit), isoamyl acetate (banana), methyl butanoate (fruity), methyl salicylate (wintergreen), octyl acetate (orange), pentyl butanoate (pear), pentyl pentanoate (apple), sotolon (curry), and “strawberry” aldehyde.

Ethers include anethole (anise), anisole, and eugenol (clove). Ketones include dihydrojasmone (floral), act-1-3-en-3-one (mushroom), 2-acetyl-1-pyrolline (bread/jasmine), and 6-acetyl-2,3,4,5-tetrahydropyridine (tortillas).

Lactones include gamma-decalactone (peach), gamma-nonalactone (coconut), delta-octalacotone (cream), jasmine lactone (fruity), massoia lactone (coconut), wine lactone (coconut), terpenes include camphor, citronellol, limonene, linalool, nerol, nerolidol, alpha-terpineol, thujone, and thymol.

Thiols include grapefruit mercaptan, methanethiol. Other components include neorolin.

The odor-masking agent may, in one or more embodiments, be sprayed onto or impregnated within packaging material. Alternatively, the odor-masking agent may be placed on an inert carrier. In yet another embodiment, the odor-masking agent may be placed inside a container adapted to diffuse the odor.

Compositions according to one or more embodiments of the present invention will be further described and advantages thereof will be made apparent with reference to the following Examples which are provided to illustrate the practice of the invention and not to limit its scope of the invention as defined by the appended claims. All percentages are by weight unless otherwise indicated.

### Examples

### Formulation of Granules Comprising Fragrance and Exemplary Fragrances

Compositions according to one or more embodiments of the present invention may be made blending the fragrance (odor-masking agent) with granulated agricultural compositions of varying particle size and bulk density. Blending may occur in a two step process wherein the granulated composition is placed into a ribbon blender and other active ingredients are added prior to the addition of the fragrance (odor-masking agent). When the fragrance comprises ingredients known as having “green notes,” more specifically (but not limited to) galbanum oil, phenyl ethyl methyl ether, phenyl acetaldehyde, phenyl acetaldehyde dimethyl acetal, cis-3-hexenyl acetate, and cinnamic alcohol, then fragrance is sprayed into the ribbon blender after addition of an actives with a nozzle to produce a composition comprising between about 0.01% to about 0.15% by weight of the total composition of fragrance, alternatively between about 0.05% to about 0.09%, and further alternatively about 0.05% or about 0.07%.

The following table indicates some preferred concentrations of an exemplary fragrance formulation according to one or more embodiments of the present invention.

TABLE 2
Exemplary Fragrance Formulation

| Compound | Amount (%) |
|---|---|
| phenyl ethyl methyl ether | 0.1%-<1% |
| galbanum oil | 0.1%-<1% |
| phenyl acetaldehyde | 1%-<5% |
| phenyl acetaldehyde | 1%-<5% |
| dimethyl acetal | |
| cis-3-hexenyl acetate | 0.1%-<1% |
| cinnamic alcohol | 0.1%-<1% |
| Other Constituents* | To 100% of |
| | Fragrance |

*May constitute additional fragrance notes, preservatives, binders, fillers, volatilizing agents, surfactants, and so on.

For example, an exemplary fragrance formulation which can be added to the agricultural composition may comprise 0.5% phenyl ethyl methyl ether, 0.25% galbanum oil, 2% phenyl acetaldehyde, 1% phenyl acetaldehyde dimethyl acetal, 0.25% cis-3-hexenyl acetate, 0.25% cinnamic alcohol, and either filler to 100% or one or more of the additional ingredients listed in Table 3 below. Table 3 indicates some preferred concentrations of ingredients. It is noted that the components listed in Table 2 are most preferred but any component in Table 3 may be added to the composition.

TABLE 3
Component Ranges

| Compound | Amount (%) |
|---|---|
| Amyl salicylate, benzyl acetate, hedione, and/or phenyl | ≧10% |
| ethyl alcohol | |
| Linalool | 5-10% |
| Amyl cinnamic aldehyde, cyclamen aldehyde, dipropylene | 1%-<5% |
| glycol, eugenol, hydroxycitronellal, phenyl acetaldehyde, | |
| phenyl acetaldehyde dimethyl acetal | |
| Benzyl benzoate, cinnamic alcohol, galbanum oil, | 0.1%-1% |
| habanolide, cis-3-hexenyl acetate, cis-3-hexenyl acetate, | |
| phenyl ethyl methyl ether, peru balsam oil, prenyl | |
| salicylate, triplal extra | |

In another example, granules of an agricultural composition are made using a drum granulation process, resulting in rounded particles (−10/+40, US sieve). Granule size may be adjusted to modulate the surface area of the granules, with larger surface area's providing improved volatilization of the fragrance. For example, a particle/granule size such that about 90% of particles/granules will pass through a 10 US sieve and be retained by a 40 US sieve provides an advantageous level of fragrance volatilization and optimum usability of the agricultural composition.

In yet another example, granules of an agricultural composition are made using a drum granulation process, again resulting in rounded particles but of a −10/+25 US sieve size. The sieve size may be adjusted from about −5 to −15 and about +15 to +45; for example, the sieve size could be −5/+15, −10/+15, −5/+20, −10/+20, −15/+20, −5/+25, −10/+25, −15/+25, and so on (not necessarily in five unit increments).

In one embodiment, the final product has a bulk density of about 46 lb/ft³ (+/−3 lb/ft³). The fragrance is added to the particles post granulation in the active ingredient blender. The location of the fragrance addition within the blender may be after the active ingredient addition. In other embodiments, after the granules have passed through all processes of the granulation system, the fragrance oil will be added in the active ingredient addition ribbon blender, post active-ingredient addition. It will be sprayed out of a tank, under pressure into the blender through a spray header equipped with a nozzle at a level of about 0.05% or about 0.07% wt/wt. The product will then pass through conveying equipment, through a scalper screen and into a hopper, where it will then be fed into the final product packaging system.

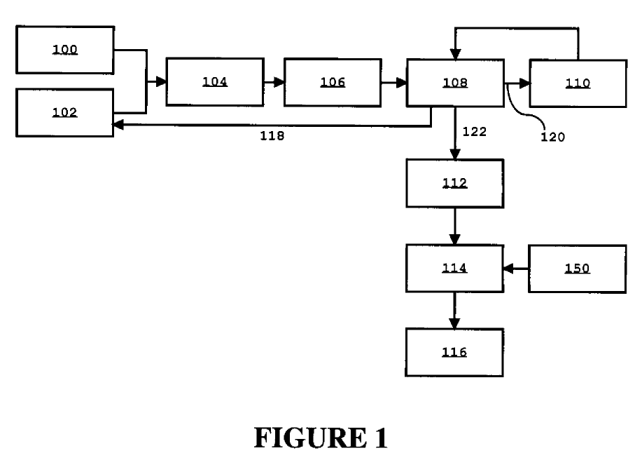

With reference to FIG. 1, an example process of making an agricultural composition according to one or more embodiments of the present invention is presented. Resin 100 and solids 102 are combined in a granulation drum 104. After processing in a process cooler 106, the preliminary agricultural composition is screened with screen 108. Oversize granules 120 are milled by mill 110, whereas undersize particles are returned to the beginning 118. On-size granules 122 are then processed by a product cooler 112, prior to addition of the fragrance 150 in a blender 114. The final product is then packaged 116.

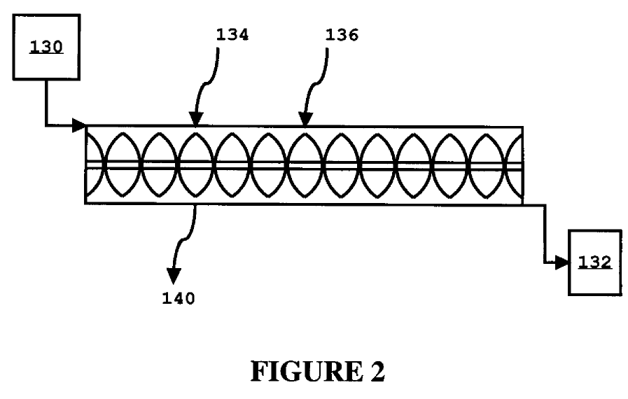

With reference to FIG. 2, an example of the blending process is presented. Agricultural composition 130 (from product cooler 112) is placed into a rotary blender 140. Active ingredient addition to the particulate or granular core occurs first, at 134, following by fragrance addition, at 136, before the product is sent to packaging 132 (to packaging 116).

A fragrance, or odor-masking agent, may comprise various amounts of galbanum oil, phenyl ethyl methyl ether, phenyl acetaldehyde, phenyl acetaldehyde dimethyl acetal, cis-3-hexenyl acetate, and cinnamic alcohol. Proportions are adjusted until maximal odor control is achieved relative to the target or odor source.

### Examples

### Efficacy Tests and Organoleptics

“Sniff-tests” are used to evaluate the efficacy of the formulation, allowing for modification of the specific proportions used in the odor-masking agent. Small glass jars containing the fragranced product, i.e. the odor-source and the odor-masking agent, are provided to persons who open the jars, smell the product, and then note whether there is a determinable phenoxy or phenolic odor present or remaining. Alternative testing may comprise the smelling of bags containing the product and odor-masking reagent. The product is placed in bags similar to those used to sell the product commercially and sealed in the normal manner. Persons then open the bags and note the smell. Testing may also comprise the noting of odor by persons as they use the product, such as by spreading the agricultural product onto a lawn.

Testing of compositions according to one or more of embodiments of the present invention which comprise the odor-masking agent on plants has demonstrated that there is no appreciable loss of activity of the product with odor-masking agent relative to the product alone.

Although the invention has been described in its preferred forms with a certain degree of particularity, it is to be understood that the present disclosure has been made by way of example only. Numerous changes in the details of the compositions and in the operational steps of the methods and in the materials utilized therein will be apparent without deporting from the spirit and scope of the invention, as defined in the appended claims.

I claim:

1. A composition, comprising
a. a dry agricultural composition having a phenolic or phenoxy-like malodor; and
b. at least one odor-masking agent in an amount effective to reduce said malodor, comprising phenyl ethyl methyl ether and optionally galbanum oil, phenyl acetaldehyde, phenyl acetaldehyde dimethyl acetal, cis-3-hexenyl acetate, cinnamic alcohol, or any combination thereof.

2. The composition of claim 1, wherein said malodor is produced at least in part by a phenoxy compound, a halophenoxy compound, or a chlorophenoxy compound.

3. The composition of claim 2, wherein said malodor is produced at least in part by (2,4-dichlorophenoxy)acetic acid.

4. The composition of claim 1, wherein said dry agricultural composition comprises at least one herbicide.

5. The composition of claim 1, wherein said dry composition produces a pleasant-smelling odor.

6. The composition of claim 1, wherein said odor-masking agent is present in an amount of about 0.05% to about 0.1% by weight of the composition.

7. The composition of claim 1, wherein said agricultural composition has an average sieve size of about −10/+40 or about −10/+25.

8. The composition of claim 1, wherein said agricultural composition comprises at least one pesticide.

9. The composition of claim 1, comprising galbanum oil, phenyl ethyl methyl ether, phenyl acetaldehyde, phenyl acetaldehyde dimethyl acetal, cis-3-hexenyl acetate, and cinnamic alcohol.

10. The composition of claim 1, wherein the odor masking agent comprises between about 0.1% to about 1% of phenyl ethyl methyl ether.

11. The composition of claim 1, comprising an inner core and an outer core, wherein said inner core comprises said agricultural composition and said outer core comprises said odor-masking agent.

12. The composition of claim 1, wherein said agricultural composition comprises a chlorophenoxy herbicide.

13. The composition of claim 12, wherein said agricultural composition comprises a fertilizer.

14. A method of making an agricultural composition with a reduced malodor impression, comprising the step of combining a granulated, dry agricultural composition with at least one odor-masking agent in an amount effective to reduce at least one phenolic or phenoxy-like odor, wherein said odor-masking agent comprises phenyl ethyl methyl ether and optionally galbanum oil, phenyl acetaldehyde, phenyl acetaldehyde dimethyl acetal, cis-3-hexenyl acetate, cinnamic alcohol, or any combination thereof.

15. The method of claim 26, wherein said odor-masking agent is coated onto said agricultural composition.

16. The method of claim 26, wherein said odor-masking agent is sprayed onto said agricultural composition.

17. The method of claim 26, wherein said malodor is phenoxy-like or phenolic.

18. The method of claim 26, wherein said malodor smells like (2,4-dichlorophenoxy)acetic acid.
